(19) United States
(12) Patent Application Publication
(10) Pub. No.: US 2024/0263112 A1
(43) Pub. Date: Aug. 8, 2024

(54) CULTURE DEVICE

(71) Applicant: HONDA MOTOR CO., LTD., Tokyo (JP)

(72) Inventors: Minoru Goto, Wako-shi (JP); Nozomi Shiobara, Wako-shi (JP); Shohei Kinoshita, Wako-shi (JP); Mizuho Doi, Wako-shi (JP); Kenji Machida, Wako-shi (JP); Kensuke Takahashi, Wako-shi (JP); Ken Harada, Wako-shi (JP); Yu Arakawa, Wako-shi (JP); Satoshi Shiozaki, Wako-shi (JP)

(21) Appl. No.: 18/432,102

(22) Filed: Feb. 5, 2024

(30) Foreign Application Priority Data
Feb. 8, 2023 (JP) 2023-017219

(51) Int. Cl. C12M 1/00; C12M 1/04; C12M 1/34; C12M 3/00

(52) U.S. Cl. CPC C12M 21/02; C12M 23/44; C12M 27/04; C12M 41/34

(57) ABSTRACT

A culture device includes an accommodation portion that accommodates a culture solution and microalgae. The accommodation portion is provided with a guide portion and a gas supply portion. The gas supply portion includes an extended tube section that extends from an upper portion to a lower portion of the guide portion in the depth direction. The extended tube portion supplies gas at the lower portion of the guide portion in the depth direction. This gas causes convection in the culture solution. The convection includes an upward flow from a lower portion to an upper portion of the accommodation portion in the depth direction. The extended tube portion is located in an upward flow region where the upward flow region is the portion through which the upward flow passes.

### CROSS-REFERENCE TO RELATED APPLICATIONS

This application is based upon and claims the benefit of priority from Japanese Patent Application No. 2023-017219 filed on Feb. 8, 2023, the contents of which are incorporated herein by reference.

### BACKGROUND OF THE INVENTION

### Field of the Invention

The present invention relates to a culture device for culturing microalgae.

### Description of the Related Art

Efforts aimed at climate change mitigation or impact reduction have continued, and toward the realization thereof, research and development in relation to the reduction of carbon dioxide emissions are being carried out. From this point of view, attention has been focused on microalgae. This is because microalgae consume carbon dioxide through photosynthesis. Therefore, a culture system for culturing microalgae is anticipated as a system that contributes to the climate change mitigation or impact reduction.

The culture system includes a culture device for culturing microalgae. As described in JP H04-370087 A and JP H08-038156 A, the culture device has an accommodation portion that accommodates culture solution and microalgae, and a gas supply portion that supplies gas into the culture solution. In many cases, the gas used is a gas containing carbon dioxide. It is to make the microalgae perform photosynthesis by supplying carbon dioxide to microalgae.

### SUMMARY OF THE INVENTION

It is preferable to agitate the culture solution in order to diffuse carbon dioxide uniformly into the culture solution. Thus, for example, it is conceivable to generate convection in the culture solution and agitate the culture solution by this convection. However, in this case, depending on the position of the gas supply portion in the culture solution, convection may be inhibited.

An object of the present invention is to solve the above-described problems.

According to one embodiment of the present invention, there is provided a culture device for culturing microalgae in a culture solution, including an accommodation portion configured to accommodate the culture solution and the microalgae; a guide portion provided inside the accommodation portion and extending in a depth direction of the accommodation portion, and a gas supply portion supplying gas at a lower portion of the guide portion in the depth direction, wherein the gas supply portion includes an extended tube portion that extends along the guide portion from an upper portion to the lower portion of the guide portion in the depth direction, convection of the culture solution is caused by the gas supplied into the culture solution, and when a region through which an upward flow of the convection passes is defined as an upward flow region wherein the upward flow moves from the lower portion of the accommodation portion in the depth direction to the upper portion of the accommodation portion in the depth direction, the extended tube portion of the gas supply portion is arranged in the upward flow region along the guide portion.

In the case of the present invention, the extended tube portion of the gas supply portion is arranged in the upward flow region, and therefore, the deposition of microalgae on the guide portion is avoided. Therefore, in the culture solution, microalgae are agitated with the convection. Moreover, the gas is sufficiently diffused and dissolved in the culture solution by the convection. Microalgae cultivation is promoted for the reasons described above.

The above and other objects, features, and advantages of the present invention will become more apparent from the following description when taken in conjunction with the accompanying drawings, in which a preferred embodiment of the present invention is shown by way of illustrative example.

### BRIEF DESCRIPTION OF THE DRAWINGS

FIG. 1 is a schematic cross-sectional view of a culture device according to an embodiment of the present invention viewed along one horizontal direction.

FIG. 2 is a schematic cross-sectional view of the culture device viewed along another horizontal direction.

FIG. 3 is a schematic perspective view of principal components of a guide portion and an extended tube portion constituting the culture device.

FIG. 4 is a cross-sectional view taken along line IV-IV in FIG. 3.

FIG. 5 is a cross-sectional view taken along line V-V in FIG. 3.

FIG. 6 is a cross-sectional view taken along line VI-VI in FIG. 3.

FIG. 7 is an enlarged view of principal parts of the extended tube portion having a terminal shape different from the terminal shapes shown in FIGS. 1 to 3.

FIG. 8 is a schematic cross-sectional view of a culture device according to an exemplary modification as viewed from the same direction as FIG. 2.

### DETAILED DESCRIPTION OF THE INVENTION

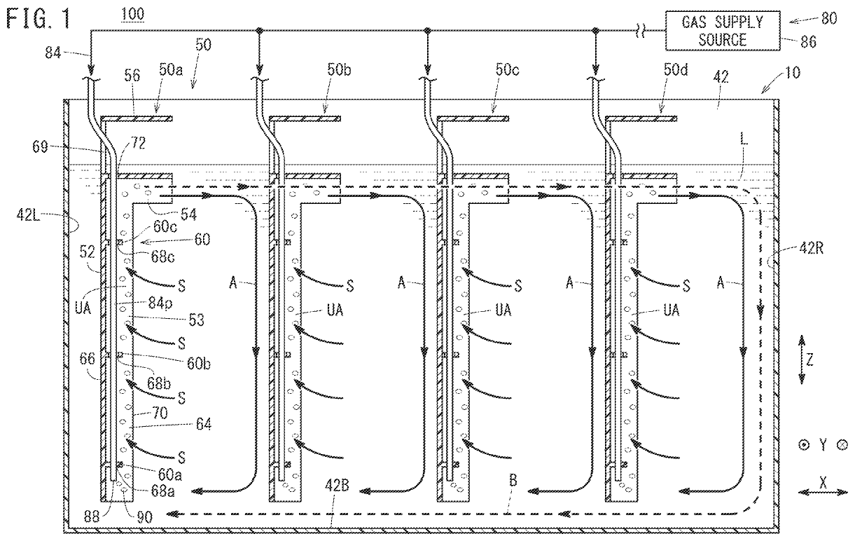

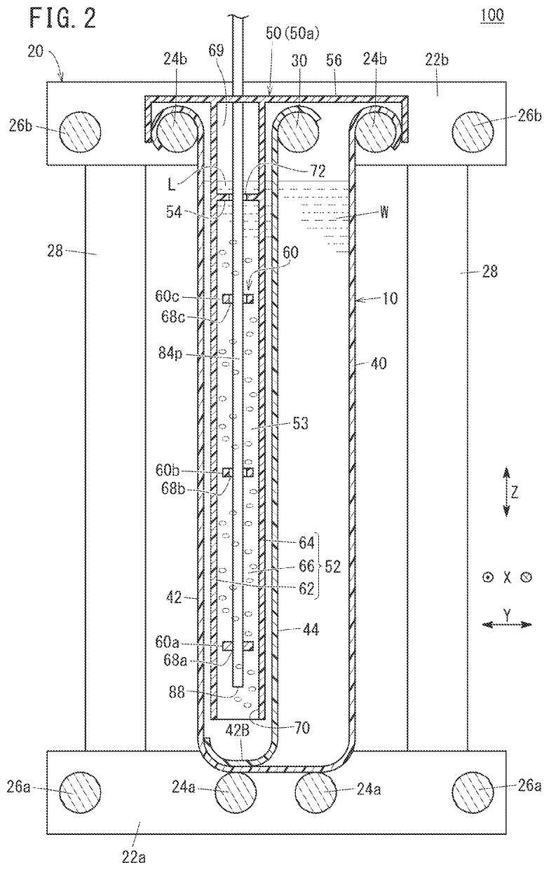

FIG. 1 is a schematic cross-sectional view of a culture device 100 according to the present embodiment as viewed along the direction of an arrow X. FIG. 2 is a schematic cross-sectional view of the culture device 100 as viewed along the direction of an arrow Y. Here, the arrow X direction and the arrow Y direction are horizontal directions orthogonal to each other. The direction of an arrow Z is the depth direction of a tank portion 10. In the aspects shown in FIGS. 1 and 2, the depth direction (arrow Z direction) of the tank portion 10 is the vertical direction. Therefore, in this aspect, the arrow Z direction is orthogonal to the arrow X and arrow Y directions.

As shown in FIGS. 1 and 2, the culture device 100 includes the tank portion 10 and a holding frame 20 (see FIG. 2) for holding the tank portion 10. First, the holding frame 20 will be briefly described. A lower portion of the holding frame 20 has two lower beam portions 22a, two lower girder portions 24a, and two reinforcing lower girder portions 26a. In FIG. 2, one of the two lower beam portions 22a is shown, and the remaining one of the two lower beam portions 22a is not shown.

Lower ends of two pillar portions 28 are joined to the lower beam portion 22a. The two lower girder portions 24a are interposed between the lower ends of the pillar portions 28 joined to one of the two lower beam portions 22a and the lower ends of the pillar portions 28 (not shown) joined to the remaining one of the two lower beam portions 22a (not shown). The two reinforcing lower girder portions 26a are located further outside than the two lower girder portions 24a.

An upper portion of the holding frame 20 has two upper beam portions 22b, two upper girder portions 24b, and two reinforcing upper girder portions 26b. Upper ends of the two pillar portions 28 are joined to one upper beam portion 22b. Similar to the lower beam portion 22a, one of the two upper beam portions 22b is shown in FIG. 2, and the remaining one of the two upper beam portions 22b is not shown.

The two upper girder portions 24b are interposed between the upper ends of the pillar portions 28 joined to one of the two upper beam portions 22b and the upper ends of the pillar portions 28 (not shown) joined to the remaining one of the two upper beam portions 22b (not shown). The two reinforcing upper girder portions 26b are located further outside than the two upper beam portions 22b. The upper portion of the holding frame 20 further has a support girder portion 30. The support girder portion 30 is located between the two upper girder portions 24b and is continuous with the two upper beam portions 22b.

The lower beam portion 22a and the upper beam portion 22b extend in the arrow Y direction. The lower girder portions 24a, the reinforcing lower girder portions 26a, the upper girder portions 24b, the reinforcing upper girder portions 26b, and the support girder portion 30 extend in the arrow X direction.

Next, the tank portion 10 will be described. As described above, in the aspects shown in FIGS. 1 and 2, the depth direction (arrow Z direction) of the tank portion 10 is the vertical direction. Therefore, in this aspect, a lower portion (downward) in the depth direction corresponds to a lower portion (downward) in the vertical direction. Similarly, an upper portion (upward) in the depth direction corresponds to an upper portion (upward) in the vertical direction.

The tank portion 10 has a water storage portion 40 and a accommodation portion 42 shown in FIG. 2. The water storage portion 40 and the accommodation portion 42 are separated by a partition portion 44. The water storage portion 40, the accommodation portion 42, and the partition portion 44 are formed of, for example, a material exhibiting flexibility and a light-transmitting property. A prime example of such a material is linear low-density polyethylene (LLDPE).

The interior of the water storage portion 40 is a space for storing the stored water W. The interior of the accommodation portion 42 is a space for accommodating microalgae and the culture solution L. As specific examples of the microalgae, there may be cited the “Honda DREAMO strain” deposited at the National Institute for Product Evaluation Technology Patent Microorganism Depositary Center. In this instance, the National Institute for Product Evaluation Technology Patent Microorganism Depositary Center is located in Room 122, 2-5-8 Kazusa Kamatari, Kisarazu City, Chiba Prefecture. The date of deposit of the Honda DREAMO strain is Apr. 22, 2016, and the accession number is FERM BP-22306. In this case, the culture solution L is typically water.

The bottom of the tank portion 10 is supported from below by the two lower girder portions 24a. In addition, first clamps (not shown) are provided on the two upper girder portions 24b, respectively. The first clamps grasp the upper edge of the accommodation portion 42 or the water storage portion 40. A second clamp (not shown) is provided on the support girder portion 30. The second clamp grasps the upper edge of the partition portion 44. In this way, the tank portion 10 is supported by the holding frame 20. The first clamps and the second clamp prevent the tank portion 10 from sagging when the stored water W is stored in the water storage portion 40 and the culture solution L is stored in the storage part 42.

As shown in FIGS. 1 and 2, a guide portion 50 is inserted into the accommodation portion 42. In this embodiment, as shown in FIG. 1, there is given an exemplary case where four guide portions 50 are provided. However, the number of guide portions 50 is not limited to four. The number of guide portions 50 may be one to three. Alternatively, the number of guide portions 50 may be four or more.

In the following, the guide portion 50 located at the leftmost position in FIG. 1 may be referred to as a first guide portion 50a. The guide portion 50 positioned to the right of the first guide portion 50a may be referred to as a second guide portion 50b, and the guide portion 50 positioned to the right of the second guide portion 50b may be referred to as a third guide portion 50c. The rightmost guide portion 50 in FIG. 1 may be referred to as a fourth guide portion 50d.

The first guide portion 50a to the fourth guide portion 50d are arranged at intervals in the width direction (arrow X direction) along the horizontal direction of the accommodation portion 42. The interval between the first guide portion 50a and the second guide portion 50b, the interval between the second guide portion 50b and the third guide portion 50c, the interval between the third guide portion 50c and the fourth guide portion 50d, and the interval between the fourth guide portion 50d and a right inner surface 42R of the accommodation portion 42 are substantially the same as each other.

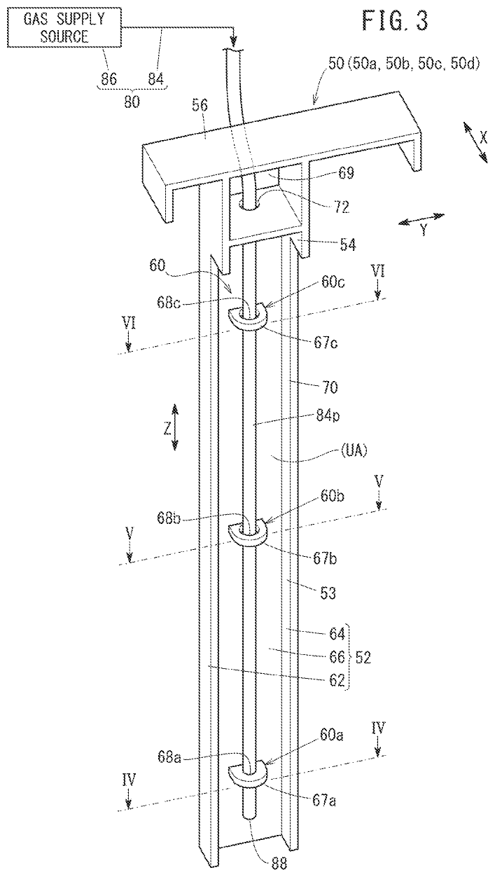

The guide portion 50 will be described with reference to FIGS. 3 to 6. As shown in FIG. 3, the guide portion 50 has a guide body 52, a deflection portion 54, a substantially inverted U-shaped held portion 56, and a support portion 60 provided on the guide body 52.

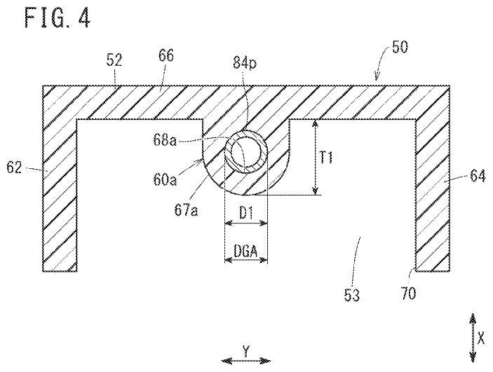

The guide body 52 extends in the depth direction (arrow Z direction/vertical direction) of the accommodation portion 42, similarly to the pillar portion 28 of the holding frame 20. In this embodiment, the guide body 52 has a first wall portion 62, a second wall portion 64, and a third wall portion 66. The first wall portion 62 and the second wall portion 64 face each other. The third wall portion 66 is connected to each of the first wall portion 62 and the second wall portion 64 so as to be approximately orthogonal to each other. Therefore, the first wall portion 62 and the second wall portion 64 are in a substantially parallel relationship. Therefore, when the guide body 52 cut along the direction orthogonal to the depth direction (the arrow X direction or the arrow Y direction) is viewed from the depth direction, a U-shape is formed by the first wall portion 62, the second wall portion 64, and the third wall portion 66 as shown in FIGS. 4 to 6.

As shown in FIG. 3, the guide body 52 has openings at the lower end in the depth direction and at one end in the extending direction. This opening forms an intake port 70 for the guide body 52. A hollow interior 53 of the guide body 52 opens toward the interior of the accommodation portion 42 via the intake port 70. The guide body 52 may be a cylindrical body only the lower end of which in the depth direction is open as the intake port 70. The guide body 52 may be formed by either one of the first wall portion 62 or the second wall portion 64 and the third wall portion 66. In this case, when the guide body 52 is cut along the arrow X direction, the cross-section of the guide body 52 seen from the arrow Z direction has a substantially L-shape.

As shown in FIGS. 1 to 6, the guide body 52 is provided with a support portion 60 on an inner surface that forms the hollow interior 53. In the illustrated example, the support portion 60 is provided on the inner surface of the third wall portion 66. However, the support portion 60 may be provided on the inner surface of the first wall portion 62 or the inner surface of the second wall portion 64.

As shown in FIGS. 1 to 3, the support portion 60 has a lowermost support portion 60a, an intermediate support portion 60b, and an uppermost support portion 60c. That is, in this embodiment, the number of support portions 60 is three. However, the number of support portions 60 is not particularly limited. The lowermost support portion 60a is located at the lowermost position in the depth direction of the guide body 52. The intermediate support portion 60b is located approximately at the middle in the depth direction of the guide body 52. The uppermost support portion 60c is located at the highest position in the depth direction of the guide body 52.

As shown in FIGS. 4 to 6, each of the lowermost support portion 60a, the intermediate support portion 60b, and the uppermost support portion 60c has a protruding portion 67a, a protruding portion 67b, and a protruding portion 67c that protrude from the inner surface of the third wall portion 66 toward the intake port 70 along the arrow X direction, respectively. A protrusion amount T1, a protrusion amount T2, and a protrusion amount T3 of the protruding portion 67a, the protruding portion 67b, and the protruding portion 67c are of sizes that can be accommodated in the hollow interior 53 of the guide body 52. In other words, the lowermost support portion 60a, the intermediate support portion 60b, and the uppermost support portion 60c are not exposed to the outside of the guide body 52 from the intake port 70. In this embodiment, the protrusion amounts T1 to T3 of the protruding portions 67a to 67c are approximately the same as each other. However, the protrusion amounts T1 to T3 may be different from each other.

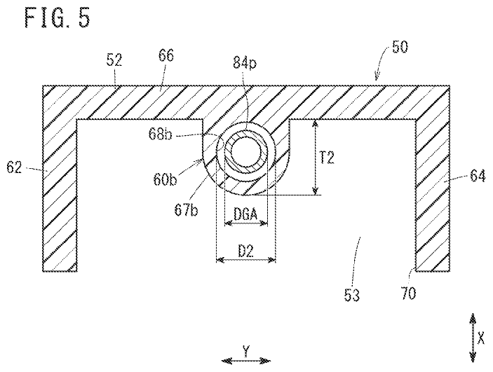

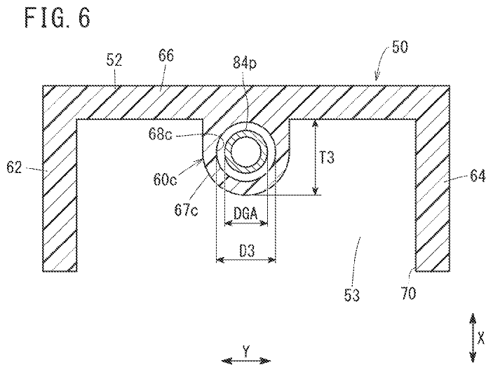

As shown in FIG. 4, the protruding portion 67a is formed with a first through hole 68a that extends in the depth direction (arrow Z direction). The diameter of the first through hole 68a is denoted as D1. As shown in FIGS. 5 and 6, the second through hole 68b and the third through hole 68c are formed in the protruding portion 67b and the protruding portion 67c, respectively. The diameters of the second through hole 68b and the third through hole 68c are denoted as D2 and D3, respectively. The diameters D2 and D3 are larger than diameter D1. Therefore, the opening area of the first through hole 68a is smaller than the opening areas of the second through hole 68b and the third through hole 68c. The diameters D2 and D3 are, for example, equal to each other. The diameters D2 and D3 may be different from each other.

The deflection portion 54 is bent by approximately 90° with respect to the depth direction at the upper end of the guide body 52 in the depth direction. The deflection portion 54 protrudes in the direction of arrow X from the upper end of the guide body 52 in the depth direction. Thereby, the deflection portion 54 is located at the upper end of the intake port 70 in the depth direction and forms the ceiling wall portion of the intake port 70. The deflection portion 54 is formed with an insertion hole 72 that extends in the depth direction. The diameter of the insertion hole 72 is slightly larger than an outer diameter DGA of the extended tube portion 84p described later and is equal to, for example, the diameters D2 and D3. The diameter of the insertion hole 72 may be smaller than the diameter D2 and the diameter D3.

As shown in FIG. 3, the lengths of the first wall portion 62 and the second wall portion 64 along the depth direction are larger than the length of the third wall portion 66 along the depth direction. The held portion 56 is provided at the upper ends of the first wall portion 62 and the second wall portion 64 in the depth direction. As shown in FIG. 2, the held portion 56 is hooked on two upper girder portions 24b of the holding frame 20. As a result of the hooking, the guide portion 50 is held by the holding frame 20.

The culture device 100 includes a gas supply device 80 shown in FIGS. 1 and 3. The gas supply device 80 has a tubular gas supply portion 84 and a gas supply source 86 connected to one end of the gas supply portion 84. The gas supply portion 84 has an extended tube portion 84p that extends along the guide portion 50. A gas discharge port 88 opens at an end portion of the extended tube portion 84p. In this aspect, the extended tube portion 84p is in the shape of a generally straight tube. Therefore, the gas discharge port 88 faces downward in the depth direction. Specifically, the gas discharge port 88 faces a bottom portion 42B of the accommodation portion 42.

As shown in FIGS. 4-6, the extended tube portion 84p has the outer diameter DGA. The diameter of the insertion hole 72, the diameter D3 of the third through hole 68c, and the diameter D2 of the second through hole 68b are larger than the outer diameter DGA. On the other hand, the diameter D1 of the first through hole 68a is approximately the same as or slightly smaller than the outer diameter DGA.

The extended tube portion 84p is positioned and fixed in the hollow interior 53 of the guide portion 50. Specifically, as shown in FIG. 3, a frame space 69 surrounded by the first wall portion 62, the second wall portion 64, and the held portion 56 is formed in the guide portion 50. The gas supply portion 84 is inserted through the frame space 69 and the insertion hole 72 formed at the deflection portion 54. The extended tube portion 84p of the gas supply portion 84 is inserted through the third through hole 68c of the uppermost support portion 60c and the second through hole 68b of the intermediate support portion 60b (see FIGS. 4 to 6). Since the diameter of the insertion hole 72 (see FIG. 3), the diameter D3 of the third through hole 68c (see FIG. 6), and the diameter D2 of the second through hole 68b (see FIG. 5) are larger than the outer diameter DGA of the extended tube portion 84p, the insertion above is easy.

As shown in FIG. 4, the diameter D1 of the first through hole 68a is approximately the same as or slightly larger than the outer diameter DGA of the extended tube portion 84p. Therefore, it is not easy to insert the extended tube portion 84p into the first through hole 68a. Therefore, this insertion is performed through, for example, light press-fitting. Therefore, it is difficult for the extended tube portion 84p to be disengaged from the first through hole 68a. It is also difficult for the extended tube portion 84p to rotate in the first through hole 68a.

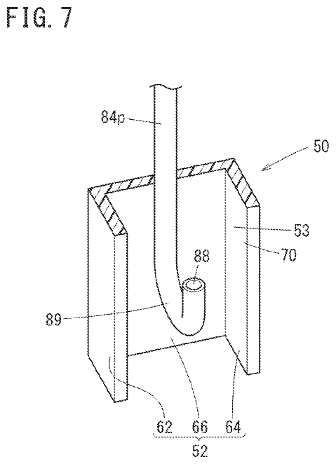

In the embodiment shown in FIGS. 1-3, the end portion of the extended tube portion 84p including the gas discharge port 88 has a substantially straight pipe shape. However, the end portion of the extended tube portion 84p may have a shape with a curved portion 89 directed upward in the depth direction as shown in FIG. 7. In this case, the gas discharge ports 88 face upward in the depth direction. Although the curved portion 89 is exaggerated in FIG. 7, it is preferable that the curved portion 89 is small enough not to hinder the convection of the culture solution L.

When the first guide portion 50a to the fourth guide portion 50d are inserted into the accommodation portion 42, all the intake ports 70 of the first guide portion 50a to the fourth guide portion 50d face in the same direction as shown in FIG. 1. Specifically, the intake port 70 of the first guide portion 50a faces the second guide portion 50b. The intake port 70 of the second guide portion 50b faces the third guide portion 50c. The intake port 70 of the third guide portion 50c faces the fourth guide portion 50d. The intake port 70 of the fourth guide portion 50d faces the right inner surface 42R of the accommodation portion 42. The third wall portion 66 of the first guide portion 50a is positioned close to the left inner surface 42L of the accommodation portion 42.

The deflection portion 54 at the first guide portion 50a protrudes toward the second guide portion 50b. The deflection portion 54 at the second guide portion 50b protrudes toward the third guide portion 50c. The deflection portion 54 at the third guide portion 50c protrudes toward the fourth guide portion 50d. The deflection portion 54 at the fourth guide portion 50d protrudes toward the right inner surface 42R of the accommodation portion 42.

The culture device 100 according to the present embodiment is basically configured as described above. Next, microalgae cultivation is described.

The holding frame 20 and the tank portion 10 are installed outdoors, for example. In this case, sunlight goes into the accommodation portion 42. It should be noted that the accommodation portion 42 may be illuminated with artificial light indoors.

In this state, microalgae cultivation is performed. Specifically, gas containing, for example, carbon dioxide is supplied from the gas supply source 86 (see FIG. 1). The gas flows through the gas supply portion 84 and is discharged from the gas discharge port 88 in the hollow interior 53 of the guide body 52 at the first guide portion 50a to the fourth guide portion 50d.

As schematically shown in FIG. 1, the gas derived from the gas supply portion 84 becomes bubbles 90. The bubbles 90 rise along the hollow interior 53 of the guide body 52. Here, the guide body 52 is formed with the intake port 70. Therefore, when the bubbles 90 rise, the culture solution L around the bubbles 90 is taken in from the intake port 70 as indicated by arrows S in FIG. 1. That is, the culture solution L around the bubbles 90 is entrained (carried along) by the bubbles 90. The entrained culture solution L rises along the guide body 52. That is, an upward flow passes through the hollow interior 53 of the guide body 52.

The deflection portion 54 is provided as a ceiling wall portion at the upper end of the intake port 70 in the depth direction. Taking the leftmost first guide portion 50a and the second guide portion 50b adjacent to the first guide portion 50a in FIG. 1 as an example, the deflection portion 54 at the first guide portion 50a protrudes toward the second guide portion 50b. Therefore, the culture solution L is guided by the guide body 52 at the first guide portion 50a by the bubbles 90 and then is directed toward the second guide portion 50b by the deflection portion 54. That is, the culture solution L flows upward in the depth direction and then flows along the horizontal direction. At this time, since the diameter of the insertion hole 72 of the deflection portion 54 is slightly larger than the outer diameter DGA of the extended tube portion 84p, most of the culture solution L flows along the horizontal direction without flowing out from the insertion hole 72 to an area above the deflection portion 54. In this way, the direction of the motion of the bubbles 90 and the culture solution L is changed by the deflection portion 54. In this way, the deflection portion 54 serves to direct the bubbles 90 (gas) and the culture solution L toward one side of the width direction in which the guide portions 50 are arranged. As a result of the above, a horizontal flow is generated at the liquid surface in the accommodation portion 42.

The bubbles 90 and the culture solution L directed toward the second guide portion 50b come into contact with the outer surface of the third wall portion 66 of the guide body 52 at the second guide portion 50b. The culture solution L descends along the outer surface of the third wall portion 66. That is, a downward flow is generated. The lowered culture solution L is entrained by means of the gas derived from the gas supply portion 84 at the first guide portion 50a as described above. Therefore, the culture solution L that has descended along the outer surface of the third wall portion 66 of the second guide portion 50b is attracted toward the first guide portion 50a.

The culture solution L attracted toward the first guide portion 50a is entrained by means of the bubbles 90 that rise in the hollow interior 53 of the first guide portion 50a. This causes the culture solution L to rise again. Based on the above process, a small amount of convection occurs between the first guide portion 50a and the second guide portion 50b as indicated by an arrow A in FIG. 1. Similarly, a small amount of convection also occurs between the second guide portion 50b and the third guide portion 50c. A small amount of convection also occurs between the third guide portion 50c and the fourth guide portion 50d. A small amount of convection also occurs between the fourth guide portion 50d and the right inner surface 42R of the accommodation portion 42.

Four of the small amounts of convection combine to generate a large amount of convection inside the accommodation portion 42 as indicated by the arrow B. At this time, since each of the first guide portion 50a to the fourth guide portion 50d have the first wall portion 62 and the second wall portion 64, the bubbles 90 and the culture solution L are prevented from leaking out of the guide body 52. As a result, the large amount of convection can be generated favorably. The small amounts of convection and the large amount of convection cause the diffusion of the bubbles 90. That is, the carbon dioxide contained in the gas diffuses throughout the culture solution L and is sufficiently dissolved in the culture solution L. Further, the microalgae are agitated by the small amounts of convection and the large amount of convection. As a result, settling or coalescing of microalgae is prevented.

Here, the upward flow of either the small amounts of convection or the large amount of convection passes through the hollow interior 53 of the guide body 52, as understood from the above. In other words, the hollow interior 53 is an upward flow region UA. Therefore, the extended tube portion 84p, the lowermost support portion 60a, the intermediate support portion 60b, and the uppermost support portion 60c in the hollow interior 53 are located in the upward flow region UA. In the upward flow area UA, microalgae ride the upward flow and rise. Therefore, the deposition or coalescing of microalgae is avoided in the extended tube portion 84p, the lowermost support portion 60a, the intermediate support portion 60b, and the uppermost support portion 60c.

For the reasons described above, microalgae are favorably cultured in the entire accommodation portion 42. The microalgae, during culturing thereof, sufficiently fix the carbon dioxide based on photosynthesis being actively carried out. Thus, carbon dioxide is consumed. For the reasons described above, carbon dioxide can be reduced by culturing microalgae in the culture device 100. As a result, the culture device 100 can contribute to climate change mitigation or impact reduction.

In addition, since microalgae can be agitated as described above, in this embodiment, it is not necessary to provide an agitation blade, a motor, and the like in the accommodation portion 42. Therefore, the stiffness of the holding frame 20 does not need to be excessively large. Therefore, it is possible to reduce the scale of the culture device 100 and to reduce the equipment investment.

The extended tube portion 84p passes through the first through hole 68a, the second through hole 68b, and the third through hole 68c, thereby being supported by the lowermost support portion 60a, the intermediate support portion 60b, and the uppermost support portion 60c. In particular, the extended tube portion 84p is fitted into the first through hole 68a through, for example, light press-fitting. This fitting prevents the extended tube portion 84p from coming out of the first through hole 68a. As described above, the extended tube portion 84p is positioned and fixed by being inserted through the first through hole 68a to the third through hole 68c. For this reason, the extended tube portion 84p, which contacts the upward flow in the hollow interior 53, is prevented from detaching from the guide body 52.

The present embodiment provides the following benefits.

A culture device 100 for culturing microalgae in culture solution L includes the accommodation portion 42 configured to accommodate the culture solution L and the microalgae, the guide portion 50 extending in the depth direction (arrow Z direction) of the accommodation portion 42 inside the accommodation portion 42, and the gas supply portion 84 supplying gas at the lower portion of the guide portion 50 in the depth direction. The gas supply portion 84 has the extended tube portion 84p. The extended tube portion 84p extends along the guide portion 50 from the upper portion to the lower portion of the guide portion 50 in the depth direction.

The gas supplied into the culture solution L causes convection (the small amounts of convection A and the large amount of convection B) in the culture solution L. A region through which an upward flow of the convection passes is defined as an upward flow region UA wherein the upward flow moves from the lower portion of the accommodation portion 42 in the depth direction toward the upper portion of the accommodation portion 42 in the depth direction. The extended tube portion 84p of the gas supply portion 84 is arranged in the upward flow region UA along the guide portion 50.

In a region where the downward flow of the convection passes, microalgae are directed toward the bottom of the accommodation portion 42. Therefore, microalgae tend to be deposited relatively easily. However, as can be understood from the above, the extended tube portion 84p is not located in the region through which the downward flow passes. Therefore, it is prevented that microalgae could be deposited on the guide portion 50.

Therefore, microalgae are agitated with the convection. Moreover, the gas is sufficiently diffused and dissolved in the culture solution L by the convection. Microalgae cultivation is promoted for the reasons described above.

When the gas contains carbon dioxide, microalgae fix a large amount of carbon dioxide when they are actively photosynthesizing. In other words, a large amount of carbon dioxide is consumed. For this reason, the culture device can contribute to climate change mitigation or impact reduction.

Furthermore, since the culture solution L is agitated as described above, it is not necessary to provide an agitation blade, a motor, and the like in the accommodation portion 42. Therefore, the stiffness of the holding frame 20 does not need to be excessively large. Therefore, it is possible to reduce the scale of the culture device 100 and to reduce the equipment investment.

The guide portion 50 has the guide body 52 that extends in the depth direction. As described above, in this embodiment, the guide body 52 has the first wall portion 62 and the second wall portion 64 facing each other, and the third wall portion 66 interposed between the first wall portion 62 and the second wall portion 64. The third wall portion 66 is continuous with and approximately perpendicular to the first wall portion 62 and the second wall portion 64. Therefore, a U-shape is formed by the first wall portion 62, the second wall portion 64, and the third wall portion 66 in a cross section when the guide body 52 is cut along a direction perpendicular to the depth direction. The hollow interior 53 of the guide body 52, which is the interior of the U-shape, is part of the upward flow region UA.

In this case, the hollow interior 53 of the guide body 52 at the guide portion 50 is the upward flow region UA. Therefore, the guide portion 50 does not block the upward flow.

The guide portion 50 has one or more support portions 60 which position and fix the extended tube portion 84p of the gas supply portion 84 to the guide body 52. One or more support portions 60 are provided on the inner surface of the third wall portion 66. Alternatively, one or more support portions 60 may be provided on the inner surface of the first wall portion 62 or the inner surface of the second wall portion 64.

The support portion 60 facilitates supporting the extended tube portion 84p to the guide portion 50. Also in this case, the support portion 60 is located in the upward flow region UA. Because microalgae are rising in the upward flow region UA, it is possible to avoid the deposition of microalgae on the support portion 60.

The one or more support portions 60 have protruding portions 67a, 67b, and 67c that protrude from the inner surface of the third wall portion 66 toward the hollow interior 53 of the guide body 52. The protruding portions 67a, 67b, and 67c are formed with the first through hole 68a, the second through hole 68b, and the third through hole 68c, respectively. The first through hole 68a, the second through hole 68b, and the third through hole 68c support the extended tube portion 84p.

By inserting the extended tube portion 84p through the first through hole 68a, the second through hole 68b, and the third through hole 68c, the gas supply portion 84 can be easily supported by the guide portion 50.

The one or more support portions 60 have the lowermost support portion 60a, the intermediate support portion 60b, and the uppermost support portion 60c that are arranged along the extending direction of the guide body 52. The opening area of the first through hole 68a formed in the lowermost support portion 60a located at the lowermost position in the depth direction is smaller than the opening area of the second through hole 68b formed in the intermediate support portion 60b and smaller than the opening area of the third through hole 68c formed in the uppermost support portion 60c.

In this configuration, the opening areas of the second through hole 68b and the third through hole 68c are set to be such sizes that the extended tube portion 84p can be inserted through, respectively. The opening area of the first through hole 68a is set to a size such that the extended tube portion 84p can be fitted through light press-fitting. In this case, it is not easy for the extended tube portion 84p to be disengaged from the first through hole 68a. This makes it easy to position and fix the gas supply portion 84 to the guide portion 50.

The extended tube portion 84p of the gas supply portion 84 has the gas discharge port 88 for discharging gas toward the accommodation portion 42. The gas discharge port 88 opens toward the bottom portion 42B of the accommodation portion 42.

According to the configuration above, the extended tube portion 84p can be formed into a substantially straight tube shape that extends linearly. That is, it is not necessary for the extended tube portion 84p to have a bent portion. Therefore, the concern that the convection of the culture solution L would inhibited by the bent portion is eliminated. Incidentally, if the extended tube portion 84p is provided with the bent portion, the size of the bent portion may be such that the bent portion does not hinder the convection of the culture solution L.

The guide portion 50 has the deflection portion 54 that directs the gas supplied from the extended tube portion 84p of the gas supply portion 84 toward the horizontal direction. The deflection portion 54 is disposed at an upper portion of the guide portion 50 in the depth direction. The deflection portion 54 has the insertion hole 72 through which the gas supply portion 84 is inserted.

The deflection portion 54 changes the flow direction of the upward flow to the horizontal direction as shown in FIG. 1. This makes it easier to create convection.

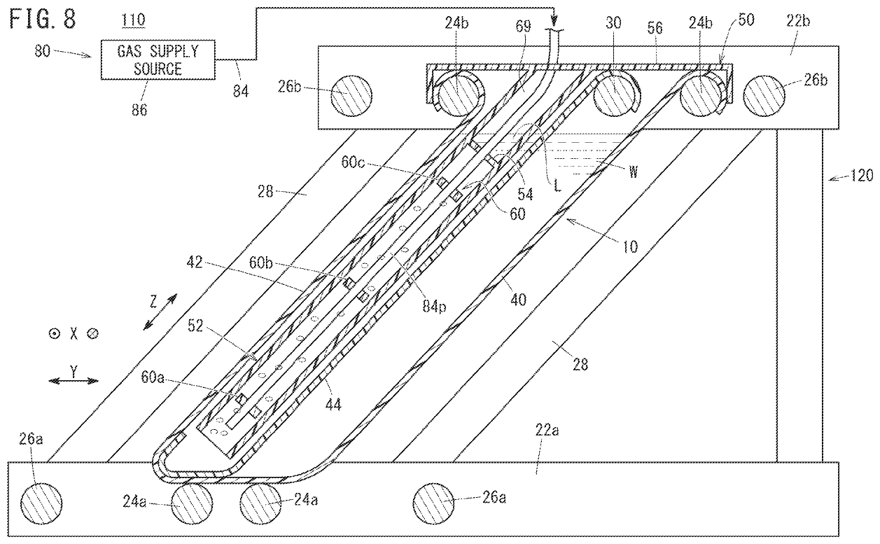

FIGS. 1 to 7 show aspects in which the depth direction (arrow Z direction) of the tank portion 10 approximately matches the vertical direction (gravity direction). In contrast, in the case of a culture device 110 shown in FIG. 8, the tank portion 10 is supported by an inclined holding frame 120. As a result, in the case of the culture device 110, the depth direction (arrow Z direction) of the tank portion 10 is inclined with respect to the vertical direction. In this aspect, an area of sunlight reception is increased. In addition, also in this aspect, the same benefits as those in the aspects shown in FIGS. 1 to 7 are obtained.

With respect to the above disclosure, the following appendixes are further disclosed.

### APPENDIX 1

A culture device (100) for culturing microalgae in a culture solution (L) include an accommodation portion (42) configured to accommodate the culture solution and the microalgae; a guide portion (50) provided inside the accommodation portion and extending in a depth direction (Z) of the accommodation portion, and a gas supply portion (84) supplying gas at a lower portion of the guide portion in the depth direction, wherein the gas supply portion includes an extended tube portion (84p) that extends along the guide portion from an upper portion to the lower portion of the guide portion in the depth direction, and convection (A, B) of the culture solution is caused by the gas supplied into the culture solution. A region through which an upward flow of the convection passes is defined as an upward flow region (UA) wherein the upward flow moves from the lower portion of the accommodation portion in the depth direction toward the upper portion of the accommodation portion in the depth direction. The extended tube portion of the gas supply portion is arranged in the upward flow region along the guide portion.

As the extended tube portion of the gas supply portion is arranged in the upward flow region, microalgae are not deposited on the guide portion. Therefore, microalgae are agitated with the convection. Moreover, the gas is sufficiently diffused and dissolved in the culture solution by the convection. Microalgae cultivation is promoted for the reasons described above.

### APPENDIX 2

In respect of the culture device described in Appendix 1, the guide portion may include a guide body (52) extending in the depth direction, the guide body may have a first wall portion (62) and a second wall portion (64) facing each other, and a third wall portion (66) interposed between the first wall portion and the second wall portion and continuous with the first wall portion and the second wall portion. A cross section of the guide body cut along a direction perpendicular to the depth direction may be formed into a U-shape by the first wall portion, the second wall portion, and the third wall portion, and a hollow interior (53) of the guide body may be part of the upward flow region.

In this case, the guide body of the guide portion is the upward flow region. Therefore, the guide portion does not block the convection.

### APPENDIX 3

In respect of the culture device according to Appendix 2, the guide portion may have one or more support portions (60) that position and fix the extended tube portion of the gas supply portion to the guide body, and the one or more support portions may be provided on the inner surface of the first wall portion, the inner surface of the second wall portion, or the inner surface of the third wall portion.

The support portion allows the extended tube portion to be easily supported by the guide portion. Also, in this case, the support portion is located in the upward flow region. Therefore, it is also possible to avoid the deposition of microalgae on the support portion.

### APPENDIX 4

In the culture apparatus described in Appendix 3, the one or more support portions may have protruding portions (67a-67c) protruding from the inner surface of the first wall portion, the inner surface of the second wall portion, or the inner surface of the third wall portion toward the hollow interior of the guide body, and through holes (68a-68c) formed in the protruding portions and extending in the depth direction of the guide body, and the through holes may support the extended tube portion of the gas supply portion.

By inserting the extended tube portion into the through hole, the gas supply portion can be easily supported by the guide portion.

### APPENDIX 5

In respect of the culture device described in Appendix 3 or 4, the one or more support portions may include a plurality of support portions (60a-60c) arranged along an extending direction of the guide body, and when a support portion of the plurality of support portions located at a lowermost position in the depth direction may be defined as a lowermost support portion (60a), an opening area of a through hole (68a) formed in the lowermost support portion may be smaller than an opening area of a through hole (68b, 68c) formed in a support portion (60b, 60c) other than the lowermost support portion.

In this configuration, for example, the opening area of the through holes formed in the support portions other than the lowermost support portion is set to a size that allows the extended tube portion to be inserted. In addition, the opening area of the through hole formed in the lowermost support portion is set to a size such that the extended tube portion can be fitted through light press-fitting, for example. Therefore, it is not easy for the extended tube portion to be disengaged from the through hole of the lowermost support portion.

As can be understood from the above, in this configuration, the extended tube portion passes through the through holes formed in the support portions other than the lowermost support portion, whereby the extended tube portion is positioned. Further, the extended tube portion is fitted into the through hole formed in the lowermost support portion, whereby it is prevented that the extended tube portion comes out from the through hole. The gas supply portion is thereby positioned and fixed. Thus, according to this configuration, it is easy to position and fix the gas supply portion to the guide portion.

### APPENDIX 6

In respect of the culture device according to any one of Appendices 1 to 5, the extended tube portion of the gas supply portion may include a gas discharge port (88) that discharges gas toward the accommodation portion, and the gas discharge port may open toward a bottom portion (42B) of the accommodation portion.

According to the above configuration, the extended tube portion can be formed into a substantially straight tube shape. That is, it is not necessary for the extended tube portion to have a bent portion. Therefore, the concern that convection of the culture solution is inhibited by the bent portion is eliminated.

### APPENDIX 7

In respect of the culture device according to any one of Appendices 1 to 6, the guide portion may include a deflection portion (54) that directs the gas supplied from the extended tube portion of the gas supply portion in a horizontal direction, and the deflection portion may be arranged at the upper portion of the guide portion in the depth direction, and the deflection portion may include an insertion hole (72) through which the gas supply portion is inserted.

The flow direction of the upward flow is changed to the horizontal direction by the deflection portion. This makes it easier to create convection.

Moreover, the gas supply portion passes through the insertion hole, whereby the extended tube portion is arranged in the upward flow region. In contrast, a portion of the gas supply portion located above the deflection portion is exposed to the outside of the culture solution through the insertion hole. Therefore, the gas supply portion is not located in a region other than the upward flow region in the culture solution. Thus, the gas supply portion does not block the convection.

The present invention is not limited to the above disclosure, and various configurations can be employed without departing from the gist of the present invention.

What is claimed is:

1. A culture device for culturing microalgae in a culture solution, the culture device comprising:
an accommodation portion configured to accommodate the culture solution and the microalgae;
a guide portion provided inside the accommodation portion and extending in a depth direction of the accommodation portion;
a gas supply portion configured to supply gas at a lower portion of the guide portion in the depth direction;
wherein
the gas supply portion includes an extended tube portion that extends along the guide portion from an upper portion to the lower portion of the guide portion in the depth direction,
convection of the culture solution is caused by the gas supplied into the culture solution, and
when a region through which an upward flow of the convection passes is defined as an upward flow region wherein the upward flow moves from a lower portion of the accommodation portion in the depth direction toward the upper portion of the accommodation portion in the depth direction, the extended tube portion of the gas supply portion is arranged in the upward flow region along the guide portion.

2. The culture device according to claim 1, wherein the guide portion includes a guide body extending in the depth direction,
the guide body includes a first wall portion and a second wall portion facing each other, and a third wall portion interposed between the first wall portion and the second wall portion and continuous with the first wall portion and the second wall portion, and a cross section of the guide body cut along a direction perpendicular to the depth direction is formed into a U-shape by the first wall portion, the second wall portion, and the third wall portion, and
a hollow interior of the guide body is part of the upward flow region.

3. The culture device according to claim 2, wherein the guide portion includes one or more support portions that position and fix the extended tube portion of the gas supply portion to the guide body, and
the one or more support portions are provided on an inner surface of the first wall portion, an inner surface of the second wall portion, or an inner surface of the third wall portion.

4. The culture device according to claim 3, wherein the one or more support portions include protruding portions protruding from the inner surface of the first wall portion, the inner surface of the second wall portion, or the inner surface of the third wall portion toward the hollow interior of the guide body, and through holes formed in the protruding portions and extending in the depth direction of the guide body, and the through holes support the extended tube portion of the gas supply portion.

5. The culture device according to claim 4, wherein the one or more support portions include a plurality of support portions arranged in an extending direction of the guide body, and when a support portion of the plurality of support portions located at a lowermost position in the depth direction is defined as a lowermost support portion, an opening area of a through hole formed in the lowermost support portion is smaller than an opening area of a through hole formed in a support portion other than the lowermost support portion.

6. The culture device according to claim 1, wherein the extended tube portion of the gas supply portion includes a gas discharge port configured to discharge gas toward the accommodation portion, and the gas discharge port opens toward a bottom portion of the accommodation portion.

7. The culture device according to claim 1, wherein the guide portion includes a deflection portion that directs the gas supplied from the extended tube portion of the gas supply portion in a horizontal direction, and the deflection portion is arranged at the upper portion of the guide portion in the depth direction, and
the deflection portion includes an insertion hole through which the gas supply portion is inserted.
